(19) United States
(12) Patent Application Publication
(10) Pub. No.: US 2018/0064628 A1
(43) Pub. Date: Mar. 8, 2018

(54) PRODUCT FOR KERATIN FIBRES, CONTAINING AT LEAST ONE COPOLYMER BASED ON ACRYLATES AND AT LEAST ONE CROSS-LINKED POLYURETHANE-VINYL-COPOLYMER

(71) Applicant: Henkel AG & Co. KGaA, Duesseldorf (DE)

(72) Inventors: Julia Bibiane Lange, Bad Bramstedt (DE); Anna Puls, Winsen (Luhe) (DE); Cyrielle Martinez, Hamburg (DE); Bernd Richters, Hamburg (DE)

(73) Assignee: Henkel AG & Co. KGaA, Duesseldorf (DE)

(21) Appl. No.: 15/561,983

(22) PCT Filed: Jan. 26, 2016
(86) PCT No.: PCT/EP2016/051569

§ 371 Date: Sep. 27, 2017

(30) Foreign Application Priority Data
Mar. 31, 2015 (DE) 10 2015 205 758.5

(51) Int. Cl. A61K 8/87; A61K 8/81; A61K 8/41; A61Q 5/06

(52) U.S. Cl. CPC A61K 8/87; A61K 8/8158; A61K 8/8152; A61K 2800/594; A61K 8/41; A61Q 5/06; A61K 8/8147

(57) ABSTRACT

The invention relates to a cosmetic product for temporary shaping keratin fibers, in particular human hair, containing a combination of copolymer A based on acrylates with a cross-linked polyurethane-vinyl copolymer B. Said cosmetic products have an extremely good level of moisture resistance. The invention also relates to the use of said cosmetic products and to a method of temporary shaping keratin fibers using said product.

### CROSS-REFERENCE TO RELATED APPLICATION

This application is a U.S. National-Stage entry under 35 U.S.C. § 371 based on International Application No. PCT/EP2016/051569, filed Jan. 26, 2016 which was published under PCT Article 21(2) and which claims priority to German Application No. 10 2015 205 758.5, filed Mar. 31, 2015, which are all hereby incorporated in their entirety by reference.

### TECHNICAL FIELD

The application relates to the technical field of temporarily reshaping keratin-containing fibers, in particular human hair. A first subject of the application is constituted by cosmetic products containing at least one copolymer A based on acrylates and at least one cross-linked polyurethane-vinyl copolymer B. Additional subjects of the present application are the use of these cosmetic products and also corresponding application methods.

### BACKGROUND

A nice-looking hairstyle is nowadays generally regarded as an essential part of a well-groomed appearance. Time and time again, based on current fashion trends, hairstyles that with many types of hair can be constructed only using firming active ingredients or that stay in place for a relatively long period of time up to several days are considered chic. Hair treatment products that permanently or temporarily shape the hair therefore play an important role. Whereas, in the case of permanent reshaping, the chemical structure of the keratin-containing fibers is modified by reduction and oxidation, no such modification of the chemical structure takes place in the case of temporary reshaping. Corresponding products for temporary shaping usually contain synthetic polymers and/or waxes as firming active ingredient.

The most important property of a product for temporarily shaping keratin fibers, also referred to hereinafter as styling products, lies in providing the treated fibers with the greatest possible hold in the newly modelled form—i.e. a form impressed on the fibers. If the keratin fibers are human hair, reference is also made to a strong hairstyle hold or to a high holding power of the styling product. The hold of a hairstyle is determined fundamentally by the type and quantity of the used firming active ingredients, however the further constituents of the styling product and also the application form can also have an effect.

In addition to a high holding power, styling products must also satisfy a wide range of further requirements. These can be divided roughly into properties on the hair, properties of the particular formulation, for example properties of the sprayed aerosol or non-aerosol, and properties that concern the handling of the styling product, wherein the properties on the hair are attributed particular importance. In particular, moisture resistance, low stickiness, and a balanced conditioning effect can be cited. Furthermore, a styling product should be universally usable for all hair types where possible and should be mild on the hair and skin.

In order to satisfy the different requirements, a multiplicity of synthetic polymers which are used in styling products have been developed in the prior art as firming active ingredients. These polymers can be divided into cationic, anionic, non-ionic and amphoteric firming polymers. When applied to the hair, the polymers ideally provide a polymer film, which on the one hand gives the hairstyle a strong hold, but on the other hand is sufficiently flexible so as not to break under load. If the polymer film is too brittle, this results in the formation of what are known as film flakes or residues, which come loose as the hair moves and give the impression that the user of the corresponding styling product has dandruff

The polymer films obtainable using the synthetic polymers and polymer combinations known in the prior art indeed demonstrate a satisfactory hold, however this attained hairstyle hold diminishes strongly in a humid environment. This compromised hold in a moist environment, however, goes against the consumer demand for a strong, consistent long-term hold irrespective of external weather influences. There is thus a need to improve the moisture resistance of polymer films obtainable by polymers and polymer combinations, but without negatively influencing the other aforementioned properties of cosmetic products, in particular styling products.

### BRIEF SUMMARY

A cosmetic product for the temporary shaping of keratin fibers is provided herein. The cosmetic product includes, in a cosmetically acceptable carrier, a) at least one copolymer A, including the following monomers a1) and a2) and a3): a1) at least one N-alkyl acrylamide, a2) at least one C₁₋₈-alkyl amino-C₂₋₈-alkyl acrylate and/or C₁₋₈-alkyl amino-C₂₋₈-alkyl(meth)acrylate, and a3) at least one further monomer, selected from acrylic acid, methacrylic acid, C₁₋₈ alkyl esters of acrylic acid and/or (meth)acrylic acid and C₂₋₈ hydroxy alkyl esters of acrylic acid and/or (meth)acrylic acid. The cosmetic product further includes b) at least one cross-linked polyurethane-vinyl copolymer B. The cosmetic product further includes c) at least one alkaline compound.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to provide new polymer combinations which, in addition to a strong long-term hold, high flexibility and low stickiness, also have an excellent moisture resistance—in particular resistance to sweat and water. Furthermore, these polymer combinations should make it possible to formulate stable viscous and also transparent cosmetic products for temporary shaping of keratin fibers.

It has now surprisingly been found that a combination of a copolymer A based on acrylates with a cross-linked polyurethane-vinyl copolymer B leads to styling products that have a strong hold and good flexibility alongside excellent moisture resistance. The combination of the aforementioned polymers surprisingly leads to a synergistic effect in respect of the moisture resistance, which was determined with the aid of the HHCR test (high humidity curl retention test). The low stickiness of the cosmetic products, which is necessary for a pleasant feel of the product, was not influenced disadvantageously by the polymer combination as contemplated herein. Furthermore, stable viscous and transparent cosmetic products can be formulated with use of this polymer combination.

A first subject of the present disclosure is therefore a cosmetic product for the temporary shaping of keratin fibers, containing, in a cosmetically acceptable carrier,
a) at least one copolymer A, comprising the following monomers a1) and a2) and a3);
a1) at least one N-alkyl acrylamide,
a2) at least one C₁₋₈-alkyl amino-C₂₋₈-alkyl acrylate and/or C₁₋₈-alkyl amino-C₂₋₈-alkyl(meth)acrylate,
a3) at least one further monomer, selected from acrylic acid, methacrylic acid, C₁₋₈ alkyl esters of acrylic acid and/or (meth)acrylic acid and C₂₋₈ hydroxy alkyl esters of acrylic acid and/or (meth)acrylic acid,
b) at least one cross-linked polyurethane-vinyl copolymer B, and
c) at least one alkaline compound.

By combining a copolymer A based on acrylates with a cross-linked polyurethane-vinyl copolymer B, a synergistic effect in respect of the improved moisture resistance of the cosmetic products was achieved. The products as contemplated herein therefore ensure a lasting and consistent strong hairstyle hold, even in the presence of changing external ambient influences. Furthermore, the use of the aforementioned polymer combination does not lead to a negative influence on the further product properties, for example the stickiness of the products. In addition, this polymer combination also does not have any negative effects on the viscosity or the desired transparency of the cosmetic products.

The term “keratin-containing fibers” is understood in principle to mean all animal hair, for example wool, horsehair, angora hair, furs, feathers and products or textiles manufactured therefrom. However, the keratin fibers are preferably human hair.

The term “cross-linked copolymers” is understood as contemplated herein to mean copolymers which have a linking of the polymer chains by a direct covalent bonding of the polymer chains or by bridging molecule fragments bonded covalently to the polymer chains. Cross-linked copolymers as contemplated herein therefore have a network formed by covalent chemical bonds.

As contemplated herein, the term “alkaline compounds” is understood to mean compounds that can cleave hydroxide ions in protic solvents or that can form salts with acids by neutralization.

In addition, the term “fatty acid” as used as contemplated herein is understood to mean aliphatic carboxylic acids which comprise unbranched or branched carbon groups having 4 to 40 carbon atoms. The fatty acids used as contemplated herein can be either naturally occurring or also synthetically produced fatty acids. The fatty acids can also be monounsaturated or polyunsaturated.

Lastly, The term “fatty alcohol” as contemplated herein is understood to mean aliphatic, monovalent, primary alcohols which comprise unbranched or branched hydrocarbon groups having from about 4 to about 40 carbon atoms. The fatty alcohols used within the scope of the invention can also be single or multiple.

As first essential constituent a), the cosmetic product as contemplated herein contains at least one copolymer A based on the monomers a1) to a3). Here, the monomers a2) and a3) can comprise C₁₋₈ alkyl groups or C₂-C₈ alkyl or hydroxy alkyl groups. Examples of such groups are methyl, ethyl, propyl, isopropyl, hydroxypropyl, butyl, sec-butyl, isobutyl, tert-butyl, hydroxybutyl, pentyl, hexyl, heptyl, and octyl groups.

The copolymer a) preferably consists of the monomers a1) and a2) and a3).

As contemplated herein, it can additionally be provided that the copolymer A contains from about 20 to about 80 wt. %, preferably from about 30 to about 50 wt. % of monomer a1) and from about 1 to about 30 wt. %, preferably from about 2 to about 10 wt. % of monomer a2), in each case in relation to the total weight of the copolymer A. Copolymers A which have the aforementioned monomer distribution lead, in combination with the cross-linked polyurethane-vinyl copolymer B, to a particularly high moisture resistance of the cosmetic products as contemplated herein.

It is advantageous as contemplated herein if the copolymer A contains specific monomers a1). It is therefore preferred if the copolymer A contains, as monomer a1), an N-alkyl acrylamide with a linear or branched C₁₋₂₀ alkyl group, preferably with a linear or branched C₂₋₁₈ alkyl group, preferably with a linear or branched C₄₋₁₆ alkyl group, in particular with a linear or branched C₆₋₁₂ alkyl group.

Particularly good results are obtained as contemplated herein when the copolymer A contains N-tert-octylacrylamide as monomer a1).

It has also proven to be advantageous as contemplated herein if the copolymer A contains specific monomers a2). It is therefore preferred as contemplated herein if the copolymer A contains, as monomer a2), a C₁₋₆-alkyl amino-C₂₋₆-alkyl acrylate and/or C₁₋₆-alkyl amino-C₂₋₆-alkyl(meth)acrylate, preferably a C₁₋₄-alkyl amino-C₂₋₄-alkyl acrylate and/or C₁₋₄-alkyl amino-C₂₋₄-alkyl(meth)acrylate, in particular tert-butylaminoethyl acrylate.

It is likewise preferred if the copolymer A contains, as monomers a3), acrylic acid and/or acrylic acid ester and/or (meth)acrylic acid ester. The copolymer A therefore preferably contains acrylic acid and/or methyl(meth)acrylate and/or hydroxypropyl acrylate as monomer(s) a3).

Particularly good results in respect of the moisture resistance of the cosmetic products as contemplated herein and thus the holding power under different external weather influences are obtained if the copolymer A contains from about 30 to about 50 wt. % of N-tert-octylacrylamide, from about 2 to about 10 wt. % of tert-butylaminoethyl acrylate, from about 10 to about 20 wt. % of acrylic acid and/or (meth)acrylic acid, from about 30 to about 40 wt. % of methyl(meth)acrylate, and from about 2 to about 10 wt. % of hydroxypropyl acrylate, in each case in relation to the total weight of the copolymer A. The use of such copolymers A in combination with the at least one cross-linked polyurethane-vinyl copolymer B leads to a synergistic effect in respect of the moisture resistance compared to the moisture resistance obtained for the individual copolymers.

A copolymer A that is very particularly preferred within the scope of this embodiment is a polymer with the INCI name Octylacrylamide/Acrylates/Butylaminoethyl Methacrylate Copolymer (CAS number 70801-07-9). Such polymers are sold for example under the trade name Amphomer by National Starch.

Preferred cosmetic products as contemplated herein contain the at least one copolymer A in a total amount of from about 0.5 to about 10 wt. %, preferably from about 1.0 to about 8.0 wt. %, preferably from about 1.0 to about 6.0 wt. %, in particular from about 1.0 to about 5.0 wt. %, in relation to the total weight of the cosmetic product. The use of these quantities of the copolymer A results in the aforementioned synergistic effect in respect of the moisture resistance in a combination with the cross-linked polyurethane-vinyl copolymer B. Furthermore, these quantities, in addition to the excellent moisture resistance, also ensure an excellent holding power of the cosmetic products as contemplated herein. In addition, with use of these quantities, both a stable viscosity of these products and a sufficient transparency of these products are ensured.

As second essential constituent b), the cosmetic product as contemplated herein contains at least one cross-linked polyurethane-vinyl copolymer B. The cross-linked polyurethane-vinyl copolymer B advantageously has an interpenetrating network of urethane and vinyl polymers.

The cross-linked polyurethane-vinyl copolymer B is preferably contained in the form of an aqueous particle dispersion having a mean particle size D₅₀ of from about 20 to about 200 nm, preferably from about 40 to about 150 nm, preferably from about 50 to about 120 nm, in particular from about 65 to about 90 nm. The mean particle size D₅₀ can be determined by way of example by dynamic light scattering (DLS) (Bergna H. E. et. al.; “Collodial Silica—Fundamentals and Application”; Surfactant science series, CRC Press, 2006, 131, pages 65 to 80).

In addition, it is preferred as contemplated herein that the cross-linked polyurethane-vinyl copolymer B contains, as polyurethane polymer, a polymer from hexylene glycol, neopentyl glycol, adipic acid, methylene diphenyl diisocyanate and dimethyl propionic acid. The polyurethane polymer obtainable from the aforementioned monomers is referred to in accordance with the INCI nomenclature as Polyurethane-2. Cross-linked polyurethane-vinyl copolymers B which contain polyurethanes from the aforementioned monomers lead, in conjunction with the copolymer A, to a particularly good moisture resistance.

As contemplated herein, it is additionally preferred if the cross-linked polyurethane-vinyl copolymer B contains, as vinyl polymer, a polymer from C₁₋₆ alkyl esters of acrylic acid and/or (meth)acrylic acid, vinyl acetate, vinyl chloride, styrene, isoprene, butadiene and acrylonitrile, in particular C₁₋₆ alkyl esters of acrylic acid and/or (meth)acrylic acid.

As contemplated herein, it is therefore very particularly preferred if the cross-linked polyurethane-vinyl copolymer contains, as vinyl polymer, a polymer from methyl(meth)acrylate. The use of methyl(meth)acrylate as vinyl polymer in the cross-linked polyurethane-vinyl copolymer B likewise leads, in conjunction with the copolymer A, to a significant increase in the moisture resistance.

A polymer that is particularly preferably used as cross-linked polyurethane-vinyl copolymer B is a polymer with the INCI name Polyurethane-2 and Polymethyl Methacrylate. This is commercially available under the name Hybridur® 875 Polymer Dispersion (Air Products and Chemicals, Inc., USA), for example as a 40 wt. % dispersion (usual properties: anionic, pH approximately 7.5 to 9.0, Brookfield viscosity approximately 50-100 cPs, particle size approximately 75-80 nm).

Preferred cosmetic products as contemplated herein contain the at least one cross-linked polyurethane-vinyl copolymer B in a total amount of from about 0.1 to about 8.0 wt. %, preferably from about 0.2 to about 6.0 wt. %, preferably from about 0.4 to about 5.0 wt. %, more preferably from about 0.8 to about 4.0 wt. %, in particular from about 1.2 to about 3.2 wt. %, in relation to the total weight of the cosmetic product. The use of the aforementioned quantities of the cross-linked polyurethane-vinyl copolymer leads, in combination with the copolymer A, to a synergistic effect in respect of the moisture resistance of the cosmetic products. Furthermore, these quantities ensure a high holding power of the cosmetic products and a sufficient flexibility. In addition, with use of the aforementioned quantities, there are no unexpected effects on the stickiness, the viscosity, and the transparency of the cosmetic products as contemplated herein.

It has proven to be advantageous for the cosmetic properties of the products as contemplated herein if the cosmetic product has a ratio by weight of the total amount of the at least one uncross-linked anionic polymer a) to the total amount of the at least one uncross-linked cationic polymer b) of from about 10:1 to about 1:30, preferably from about 5:1 to about 1:30, preferably from about 1:1 to about 1:30, more preferably from about 1:1.5 to about 1:20, in particular from about 1:2 to about 1:15.

The polymers A and/or B are used in the cosmetic products as contemplated herein in partially neutralised or neutralised form. These products therefore contain at least one alkaline compound c) for neutralization.

In particular, it has proven to be advantageous as contemplated herein to use, as alkanolamines, primary amines having a C₂-C₆ alkyl parent substance carrying at least one hydroxyl group. The alkaline compound c) is therefore preferably selected from the group formed from 2-aminoethan-1-ol (monoethanolamine), tris(2-hydroxyethyl)-amine (triethanolamine), 3-aminopropan-1-ol, 4-aminobutan-1-ol, -aminopentan-1-ol, 1-aminopropan-2-ol, 1-aminobutan-2-ol, 1-aminopentan-2-ol, 1-aminopentan-3-ol, 1-aminopentan-4-ol, 2-amino-2-methylpropan-1-ol, 1-amino-2-methylpropan-2-ol, 3-aminopropan-1,2-diol, 2-amino-2-methylpropan-1,3-diol, in particular 2-amino-2-methylpropan-1-ol. Cosmetic products that are particularly preferred as contemplated herein therefore contain 2-amino-2-methylpropanol as alkaline compound c).

The alkaline compound c) is preferably used in a quantity that does not exceed the quantity required for neutralization of the copolymers A and/or B. The quantity of alkaline compound c) used in the compositions as contemplated herein is preferably from about 80 to 100%, particularly preferably from about 90 to 100%, and in particular from about 95 to 100% of the quantity required for full neutralization of the copolymers A and/or B. In a preferred embodiment the alkaline compound c) is therefore contained in a total quantity of from about 0.05 to about 7.0 wt. %, preferably from about 0.1 to about 5.0 wt. %, preferably from about 0.1 to about 4.0 wt. %, in particular from about 0.1 to about 3.0 wt. %, in relation to the total weight of the cosmetic product.

The cosmetic products as contemplated herein contain the copolymer A, the cross-linked polyurethane-vinyl polymer B, the alkaline compound c) and also optionally further ingredients, in a cosmetically acceptable carrier.

Preferred cosmetically acceptable carriers are aqueous, alcoholic or aqueous-alcoholic media comprising preferably at least about 10 wt. % water, in relation to the total weight of the cosmetic product.

The cosmetically acceptable carrier particularly preferably contains water, in particular in such a quantity that the cosmetic product, calculated on the total weight of the cosmetic product, contains at least about 10 wt. %, preferably at least about 20 wt. %, in particular at least about 40 wt. % water. Very particularly preferred cosmetic products however, in relation to their total weight, have a water content of from about 50 to about 95 wt. %, preferably from about 60 to about 90 wt. %, in particular from about 65 to about 85 wt. %.

The lower alcohols conventionally used for cosmetic purposes having 1 to 4 carbon atoms, such as ethanol and isopropanol, can be contained as alcohols, in particular.

Examples of water-soluble solvents as co-solvent are glycerol and/or ethylene glycol and/or 1,2-propylene glycol, which can be used in a quantity of from 0 to about 30 wt. %, in relation to the total weight of the cosmetic product.

The long-term hold of the cosmetic product as contemplated herein can be further increased if said product contains at least one further film-forming and/or firming polymer which is different from the copolymer A and the cross-linked polyurethane-vinyl copolymer B. However, it can also be preferred to use exclusively the aforementioned copolymer A and the aforementioned cross-linked polyurethane-vinyl copolymer B as film-forming and/or firming polymers.

Film-forming or firming polymers contribute, by the formation of a film, to the hold of the impressed form of the collective body of fibers, for example the hairstyle as a whole. The film formation can be provided at quite specific points and may connect only some fibers to one another. Film-forming or firming polymers are to be understood to mean those polymers which, as they dry, leave behind a continuous film on the skin, the hair, or the nails. Film formers of this type can be used in the widest range of different cosmetic products, such as face masks, make-up, hair setting agents, hairsprays, hair gels, hair waxes, hair masks, shampoos, or nail varnishes. In particular, polymers which have a solubility in water or water/alcohol mixtures sufficient to be present in fully dissolved form in the cosmetic products as contemplated herein are preferred. The film-forming polymers can be of synthetic or natural origin. Film-forming polymers are also understood to be polymers which, when used in 0.01 to 20 wt. % aqueous, alcoholic or aqueous-alcoholic solution, are capable of being deposited on the hair in a transparent polymer film.

In this regard, it can be provided in particular as contemplated herein that the further film-forming and/or firming polymer is selected from copolymers of polyvinylpyrrolidone with vinyl acetate. In particular, polyvinylpyrrolidone/vinyl acetate copolymers with a molar ratio of polyvinylpyrrolidone to vinyl acetate of from about 70 to about 30, from about 60 to about 40, from about 50 to about 50, or from about 30 to about 70 can be used within the scope of the present disclosure. PVP/VA copolymers of this type having a molar ratio of polyvinylpyrrolidone to vinyl acetate of from about 70 to about 30 are obtainable for example under the trade names PVP/VA E-735, PVP/VA 1-735 and PVP/VA W-735 as 50% dispersion in ethanol, isopropanol or water from the company Ashland. PVP/VA copolymers having a molar ratio of polyvinylpyrrolidone to vinyl acetate of from about 60 to about 40 are obtainable for example under the trade names PVP/VA E-635 and PVP/VA W-635 as 50% dispersion in ethanol, isopropanol or water from the company Ashland. PVP/VA copolymers having a molar ratio of polyvinylpyrrolidone to vinyl acetate of from about 50 to about 50 as 50% dispersion in ethanol or isopropanol are obtainable from the company Ashland under the trade names PVP/VA E-535 and PVP/VA 1-535. A PVP/VA copolymer having a molar ratio of polyvinylpyrrolidone to vinyl acetate of from about 30 to about 70 is sold for example under the trade name PVP/VA 1-335 as 50% dispersion in isopropanol by the company Ashland.

Besides the copolymers of polyvinylpyrrolidone and vinyl acetate, the cosmetic products as contemplated herein can contain further film-forming and/or firming polymers selected from the group of polymers of vinylpyrrolidone, copolymers of vinylpyrrolidone with vinylimidazole and/or acrylamide and/or (meth)acrylamide, copolymers of isobutene, and mixtures thereof. These polymers are also different from the copolymer A and the cross-linked polyurethane-vinyl polymer B.

These film-forming and/or firming polymers are in turn preferably selected from at least one polymer from the group formed from polyvinylpyrrolidone,

copolymers of N-vinylpyrrolidone and N-vinylimidazole and (meth)acrylamide

copolymers of N-vinylpyrrolidone and N-vinylimidazole and acrylamide,

copolymers of N-vinylpyrrolidone with N,N-Di-C₁-C₄-alkyl amino-C₂-C₄-alkyl acrylamide,

copolymers of N-vinylpyrrolidone with N,N-Di-C₁-C₄)-alkyl amino-C₂-C₄-alkyl acrylamide,

copolymers of isobutene.

Suitable polyvinylpyrrolidones are, for example, trade products such as Luviskol® K 90 or Luviskol® K 85 from the company BASF SE. Suitable polyvinyl alcohols are sold for example under the trade names Elvanol® by Du Pont or Vinol® 523/540 by the company Air Products.

The further film-forming and/or firming polymers can be contained in the cosmetic products as contemplated herein preferably in a total amount of from about 0.1 wt. % to about 12.0 wt. %, preferably from about 0.2 wt. % to about 10.0 wt. %, in particular from about 0.5 wt. % to about 8.0 wt. %, in each case in relation to the total weight of the cosmetic product.

As contemplated herein, the cosmetic composition is provided in the form of a gel. The term “gel” is understood, as contemplated herein, to mean a dimensionally stable and easily deformable system formed from two components, wherein one component, in the form of the thickening agent or gel former, forms a physical, three-dimensional network, in the spaces or pores of which the second component is incorporated in the form of liquid, in particular water.

In order to achieve the gel-like consistency, a further one or more components acting as thickening agent or gel former and different from the copolymer A and cross-linked polyurethane-vinyl copolymer B is/are advantageously used. The total quantity of this thickening agent in the total weight of the cosmetic product is, for example, from about 0.02 to about 3 wt. %, preferably from about 0.05 to about 1.5 wt. %, in particular from about 0.2 to about 0.8 wt. %.

Examples are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates/C₁₋₂ Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C₁₋₁₈ Alkyl Acrylates/C₁₋₈ Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-1/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/Vinyl Caprolactam/DMAPA Acrylates Copolymer, Yeast Palmitate and Styrene/VP Copolymer.

The thickening agent or the gel former is preferably contained in a total amount of from about 0.02 to about 3 wt. %, preferably from about 0.05 to about 1.5 wt. %, in particular from about 0.2 to about 0.8 wt. %, in relation to the total weight of the cosmetic product.

A thickening agent that is used with particular preference as contemplated herein is a homopolyacrylic acid (INCI: Carbomer), which is commercially obtainable under the name Carbopol® in various forms.

Particularly preferred embodiments (A)-(H) of the cosmetic products as contemplated herein will be detailed hereinafter:

### (A):

A cosmetic product for the temporary shaping of keratin fibers, containing, in a cosmetically acceptable carrier—in relation to its total weight—
a) from about 0.5 to about 10 wt. % of at least one above-described copolymer A
b) from about 0.1 to about 8.0 wt. % of at least one above-described cross-linked polyurethane-vinyl copolymer B,
c) from about 0.05 to about 7.0 wt. % of at least one above-described alkaline compound c),
d) 0 to about 10 wt. % of at least one above-described film-forming and/or firming polymer different from the copolymer A and the cross-linked polyurethane-vinyl polymer B, and
e) 0 to about 3 wt. % of at least one above-described thickening agent or gel former.

### (B):

A cosmetic product for the temporary shaping of keratin fibers, containing, in a cosmetically acceptable carrier—in relation to its total weight—
a) from about 0.5 to about 10 wt. %, preferably from about 1.0 to about 8.0 wt. %, preferably from about 1.0 to about 6.0 wt. %, in particular from about 1.0 to about 5.0 wt. % of at least one copolymer A, containing at least one N-alkyl acrylamide, at least one C₁₋₈-alkyl amino-C₂₋₈-alkyl acrylate and/or C₁₋₈-alkyl amino-C₂₋₈-alkyl(meth)acrylate and at least one further monomer, selected from acrylic acid, methacrylic acid, C₁₋₈ alkyl esters of acrylic acid and/or (meth)acrylic acid and C₂₋₈ hydroxy alkyl esters of acrylic acid and/or (meth)acrylic acid,
b) from about 0.1 to about 8.0 wt. %, preferably from about 0.2 to about 6.0 wt. %, preferably from about 0.4 to about 5.0 wt. %, more preferably from about 0.8 to about 4.0 wt. %, in particular from about 1.2 to about 3.2 wt. % of at least one cross-linked polyurethane-vinyl copolymer B, containing, as polyurethane polymer, a polymer from hexylene glycol, neopentyl glycol, adipic acid, methylene diphenyl diisocyanate and dimethyl propionic acid, and containing, as vinyl polymer, a polymer from C₁₋₆ alkyl esters of acrylic acid and/or (meth)acrylic acid, vinyl acetate, vinyl chloride, styrene, isoprene, butadiene and acrylonitrile, in particular C₁₋₆ alkyl esters of acrylic acid and/or (meth)acrylic acid,
c) from about 0.05 to about 7.0 wt. %, preferably from about 0.1 to about 5.0 wt. %, preferably from about 0.1 to about 4.0 wt. %, in particular from about 0.1 to about 3.0 wt. % of at least one alkaline compound c) from the group formed from 2-aminoethan-1-ol (monoethanolamine), tris(2-hydroxyethyl)-amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 2-amino-2-methylpropan-1-ol, 1-amino-2-methylpropan-2-ol, 3-aminopropan-1,2-diol, 2-amino-2-methylpropan-1,3-diol, in particular 2-amino-2-methylpropan-1-ol,
d) 0 to about 10 wt. %, preferably from about 2.0 to about 8.5 wt. %, in particular from about 3.0 to about 7.0 wt. % of at least one vinylpyrrolidone polymer and/or vinylpyrrolidone-vinyl acetate copolymer, and
e) 0 to about 3 wt. %, preferably from about 0.05 to about 1.5 wt. %, in particular from about 0.2 to about 0.8 wt. % of a homopolyacrylic acid.

### (C):

A cosmetic product for the temporary shaping of keratin fibers, containing, in a cosmetically acceptable carrier—in relation to its total weight—
a) from about 1.0 to about 6.0 wt. % of at least one copolymer A, containing at least one N-alkyl acrylamide, at least one C₁₋₈-alkyl amino-C₂₋₈-alkyl acrylate and/or C₁₋₈-alkyl amino-C₂₋₈-alkyl(meth)acrylate and at least one further monomer, selected from acrylic acid, methacrylic acid, C₁₋₈ alkyl esters of acrylic acid and/or (meth)acrylic acid and C₂₋₈ hydroxy alkyl esters of acrylic acid and/or (meth)acrylic acid,
b) from about 0.4 to about 5.0 wt. % of at least one cross-linked polyurethane-vinyl copolymer B, containing, as polyurethane polymer, a polymer from hexylene glycol, neopentyl glycol, adipic acid, methylene diphenyl diisocyanate and dimethyl propionic acid, and containing, as vinyl polymer, a polymer from C₁₋₆ alkyl esters of acrylic acid and/or (meth)acrylic acid, vinyl acetate, vinyl chloride, styrene, isoprene, butadiene and acrylonitrile, in particular C₁₋₆ alkyl esters of acrylic acid and/or (meth)acrylic acid,
c) from about 0.1 to about 4.0 wt. % of at least one alkaline compound c) from the group formed from 2-aminoethan-1-ol (monoethanolamine), tris(2-hydroxyethyl)-amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 2-amino-2-methylpropan-1-ol, 1-amino-2-methylpropan-2-ol, 3-aminopropan-1,2-diol, 2-amino-2-methylpropan-1,3-diol, in particular 2-amino-2-methylpropan-1-ol,
d) 0 to about 8.5 wt. % of at least one vinylpyrrolidone polymer and/or vinylpyrrolidone-vinyl acetate copolymer, and
e) 0 to about 1.5 wt. % of a homopolyacrylic acid.

### (D):

A cosmetic product for the temporary shaping of keratin fibers, containing, in a cosmetically acceptable carrier—in relation to its total weight—
a) from about 1.0 to about 5.0 wt. % of at least one copolymer A, containing at least one N-alkyl acrylamide, at least one C₁₋₈-alkyl amino-C₂₋₈-alkyl acrylate and/or C₁₋₈-alkyl amino-C₂₋₈-alkyl(meth)acrylate and at least one further monomer, selected from acrylic acid, methacrylic acid, C₁₋₈ alkyl esters of acrylic acid and/or (meth)acrylic acid and C₂₋₈ hydroxy alkyl esters of acrylic acid and/or (meth)acrylic acid,
b) from about 1.2 to about 3.2 wt. % of at least one cross-linked polyurethane-vinyl copolymer B, containing, as polyurethane polymer, a polymer from hexylene glycol, neopentyl glycol, adipic acid, methylene diphenyl diisocyanate and dimethyl propionic acid, and containing, as vinyl polymer, a polymer from C₁₋₆ alkyl esters of acrylic acid and/or (meth)acrylic acid, vinyl acetate, vinyl chloride, styrene, isoprene, butadiene and acrylonitrile, in particular C₁₋₆ alkyl esters of acrylic acid and/or (meth)acrylic acid,
c) from about 0.1 to about 4 wt. % of at least one alkaline compound c) from the group formed from 2-aminoethan-1-ol (monoethanolamine), tris(2-hydroxyethyl)-amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 2-amino-2-methylpropan-1-ol, 1-amino-2-methylpropan-2-ol, 3-aminopropan-1,2-diol, 2-amino-2-methylpropan-1,3-diol, in particular 2-amino-2-methylpropan-1-ol,
d) 0 to about 7.0 wt. % of at least one vinylpyrrolidone polymer and/or vinylpyrrolidone-vinyl acetate copolymer, and
e) 0 to about 0.8 wt. % of a homopolyacrylic acid.

### (E):

A cosmetic product for the temporary shaping of keratin fibers, containing, in a cosmetically acceptable carrier—in relation to its total weight—
a) from about 0.5 to about 10 wt. %, preferably from about 1.0 to about 8.0 wt. %, preferably from about 1.0 to about 6.0 wt. %, in particular from about 1.0 to about 5.0 wt. % of at least one copolymer A, containing N-tert-octylacrylamide, tert-butylaminoethyl acrylate and acrylic acid and/or methyl(meth)acrylate and/or hydroxypropyl acrylate
b) from about 0.1 to about 8.0 wt. %, preferably from about 0.2 to about 6.0 wt. %, preferably from about 0.4 to about 5.0 wt. %, more preferably from about 0.8 to about 4.0 wt. %, in particular from about 1.2 to about 3.2 wt. % of at least one cross-linked polyurethane-vinyl copolymer B, containing, as polyurethane polymer, a polymer from hexylene glycol, neopentyl glycol, adipic acid, methylene diphenyl diisocyanate and dimethyl propionic acid, and containing, as vinyl polymer, a polymer from methyl(meth)acrylate,
c) from about 0.05 to about 7.0 wt. %, preferably from about 0.1 to about 5.0 wt. %, preferably from about 0.1 to about 4.0 wt. %, in particular from about 0.1 to about 3.0 wt. % of at least one alkaline compound c) in the form of 2-amino-2-methylpropan-1-ol,
d) 0 to about 10 wt. %, preferably from about 2.0 to about 8.5 wt. %, in particular from about 3.0 to about 7.0 wt. % of at least one vinylpyrrolidone polymer and/or vinylpyrrolidone-vinyl acetate copolymer, and
e) 0 to about 3 wt. %, preferably from about 0.05 to about 1.5 wt. %, in particular from about 0.2 to about 0.8 wt. % of a homopolyacrylic acid.

### (F):

A cosmetic product for the temporary shaping of keratin fibers, containing, in a cosmetically acceptable carrier—in relation to its total weight—
a) from about 1.0 to about 6.0 wt. % of at least one copolymer A, containing N-tert-octylacrylamide, tert-butylaminoethyl acrylate and acrylic acid and/or methyl(meth)acrylate and/or hydroxypropyl acrylate
b) from about 0.8 to about 4.0 wt. % of at least one cross-linked polyurethane-vinyl copolymer B, containing, as polyurethane polymer, a polymer from hexylene glycol, neopentyl glycol, adipic acid, methylene diphenyl diisocyanate and dimethyl propionic acid, and containing, as vinyl polymer, a polymer from methyl(meth)acrylate,
c) from about 0.1 to about 4.0 wt. % of at least one alkaline compound c) in the form of 2-amino-2-methylpropan-1-ol,
d) 0 to about 8.5 wt. % of at least one vinylpyrrolidone polymer and/or vinylpyrrolidone-vinyl acetate copolymer, and
e) 0 to about 1.5 wt. % of a homopolyacrylic acid.

### (G):

A cosmetic product for the temporary shaping of keratin fibers, containing, in a cosmetically acceptable carrier—in relation to its total weight—
a) from about 0.5 to about 10 wt. %, preferably from about 1.0 to about 8.0 wt. %, preferably from about 1.0 to about 6.0 wt. %, in particular from about 1.0 to about 5.0 wt. % of at least one copolymer A, containing N-tert-octylacrylamide, tert-butylaminoethyl acrylate and acrylic acid and/or methyl(meth)acrylate and/or hydroxypropyl acrylate
b) from about 0.1 to about 8.0 wt. %, preferably from about 0.2 to about 6.0 wt. %, preferably from about 0.4 to about 5.0 wt. %, more preferably from about 0.8 to about 4.0 wt. %, in particular from about 1.2 to about 3.2 wt. % of at least one cross-linked polyurethane-vinyl copolymer B, containing, as polyurethane polymer, a polymer from hexylene glycol, neopentyl glycol, adipic acid, methylene diphenyl diisocyanate and dimethyl propionic acid, and containing, as vinyl polymer, a polymer from methyl(meth)acrylate,
c) from about 0.05 to about 7.0 wt. %, preferably from about 0.1 to about 5.0 wt. %, preferably from about 0.1 to about 4.0 wt. %, in particular from about 0.1 to about 3.0 wt. % of at least one alkaline compound c) in the form of 2-amino-2-methylpropan-1-ol,
d) 0 to about 10 wt. %, preferably from about 2.0 to about 8.5 wt. %, in particular from about 3.0 to about 7.0 wt. % of at least one vinylpyrrolidone polymer and/or vinylpyrrolidone-vinyl acetate copolymer, and
e) 0 to about 3 wt. %, preferably from about 0.05 to about 1.5 wt. %, in particular from about 0.2 to about 0.8 wt. % of a homopolyacrylic acid.

### (H):

A cosmetic product for the temporary shaping of keratin fibers, containing, in a cosmetically acceptable carrier—in relation to its total weight—
a) from about 1.0 to about 6.0 wt. % of at least one copolymer A, containing N-tert-octylacrylamide, tert-butylaminoethyl acrylate and acrylic acid and/or methyl(meth)acrylate and/or hydroxypropyl acrylate
b) from about 0.8 to about 4.0 wt. % of at least one cross-linked polyurethane-vinyl copolymer B, containing, as polyurethane polymer, a polymer from hexylene glycol, neopentyl glycol, adipic acid, methylene diphenyl diisocyanate and dimethyl propionic acid, and containing, as vinyl polymer, a polymer from methyl(meth)acrylate,
c) from about 0.1 to about 4.0 wt. % of at least one alkaline compound c) in the form of 2-amino-2-methylpropan-1-ol,
d) 0 to about 8.5 wt. % of at least one vinylpyrrolidone polymer and/or vinylpyrrolidone-vinyl acetate copolymer, and
e) 0 to about 1.5 wt. % of a homopolyacrylic acid.

### (I):

A cosmetic product for the temporary shaping of keratin fibers, containing, in a cosmetically acceptable carrier—in relation to its total weight—
a) from about 1.0 to about 5.0 wt. % of at least one copolymer A, containing N-tert-octylacrylamide, tert-butylaminoethyl acrylate and acrylic acid and/or methyl(meth)acrylate and/or hydroxypropyl acrylate
b) from about 1.2 to about 3.2 wt. % of at least one cross-linked polyurethane-vinyl copolymer B, containing, as polyurethane polymer, a polymer from hexylene glycol, neopentyl glycol, adipic acid, methylene diphenyl diisocyanate and dimethyl propionic acid, and containing, as vinyl polymer, a polymer from methyl(meth)acrylate,
c) from about 0.1 to about 3.0 wt. % of at least one alkaline compound c) in the form of 2-amino-2-methylpropan-1-ol,
d) 0 to about 7.0 wt. % of at least one vinylpyrrolidone polymer and/or vinylpyrrolidone-vinyl acetate copolymer, and
e) 0 to about 0.8 wt. % of a homopolyacrylic acid.

### (J):

A cosmetic product for the temporary shaping of keratin fibers, containing, in a cosmetically acceptable carrier—in relation to its total weight—
a) from about 1.0 to about 5.0 wt. % of at least one copolymer A, containing from about 30 to about 50 wt. % of N-tert-octylacrylamide, 2 to 10 wt. % of tert-butylaminoethyl acrylate, from about 10 to about 20 wt. % of acrylic acid and/or (meth)acrylic acid, from about 30 to about 40 wt. % of methyl(meth)acrylate, and from about 2 to about 10 wt. % of hydroxypropyl acrylate, in each case in relation to the total weight of the copolymer A.
b) from about 1.2 to about 3.2 wt. % of at least one cross-linked polyurethane-vinyl copolymer B, containing, as polyurethane polymer, a polymer from hexylene glycol, neopentyl glycol, adipic acid, methylene diphenyl diisocyanate and dimethyl propionic acid, and containing, as vinyl polymer, a polymer from methyl(meth)acrylate,
c) from about 0.1 to about 3.0 wt. % of at least one alkaline compound c) in the form of 2-amino-2-methylpropan-1-ol,
d) 0 to about 7.0 wt. % of at least one vinylpyrrolidone polymer and/or vinylpyrrolidone-vinyl acetate copolymer, and
e) 0 to about 0.8 wt. % of a homopolyacrylic acid.

The aforementioned particularly preferred embodiments (A)-(J) of the cosmetic products as contemplated herein are exemplified by an excellent moisture resistance and by a good long-term hold. Furthermore, the polymer films formed from the cited polymer combinations have sufficient flexibility, such that the formation of film flakes is avoided. In addition, these products are exemplified by a stable viscosity and a high transparency.

Besides the previously described components, the cosmetic products as contemplated herein can also contain further ingredients. This group of further ingredients includes, in particular, cosmetically effective auxiliaries and additives, in particular additional nourishing ingredients.

By way of example, a silicone oil and/or a silicone gum can be used as nourishing ingredient. Silicone oils or silicone gums that are suitable as contemplated herein are, in particular, dialkyl and alkylaryl siloxanes, such as dimethylpolysiloxane and methylphenyl polysiloxane, and also the alkoxylated, quaternised, or also anionic derivatives thereof. Cyclic and linear polydialkyl siloxanes, the alkoxylated and/or aminated derivatives thereof, dihydroxy polydimethylsiloxanes and polyphenylalkylsiloxanes, in particular PEG-12 dimethicones and PEG-14 dimethicones, are preferred.

The cosmetic product as contemplated herein can contain, for example, at least one protein hydrolysate and/or a derivative thereof as a nourishing ingredient of another compound class. Protein hydrolysates are product mixtures which are obtained by acid-catalysed, base-catalysed or enzymatically catalysed degradation of proteins (albumins). The term “protein hydrolysates” is understood as contemplated herein to also mean total hydrolysates from individual amino acids and derivatives thereof as well as mixtures of different amino acids. The molecular weight of the protein hydrolysates usable as contemplated herein lies between about 75, the molecular weight for glycine, and about 200,000 daltons, and the molecular weight is preferably from about 75 to about 50,000 daltons, in particular from about 75 to about 20,000 daltons.

As nourishing ingredient, the cosmetic composition as contemplated herein can also contain at least one vitamin, a provitamin, a vitamin precursor and/or derivatives thereof. Here, vitamins, provitamins and vitamin precursors that are usually assigned to the groups A, B, C, E, F and H are preferred as contemplated herein.

The addition of panthenol can the flexibility of the polymer film formed with application of the cosmetic product as contemplated herein.

As nourishing ingredient, the cosmetic products as contemplated herein can also contain at least one plant extract, but also monosaccharides or oligosaccharides and/or lipids.

Oil bodies are also suitable as nourishing ingredient. Natural and synthetic cosmetic oil bodies include, for example, plant oils, liquid paraffin oils, isoparaffin oils and synthetic hydrocarbons as well as di-n-alkyl ethers having a total of between 12 and 36 C atoms, in particular 12 to 24 C atoms.

Ester oils, i.e. esters of C₆-C₃₀ fatty acids with C₂-C₃₀ fatty alcohols, preferably monoesters of the fatty acids with alcohols having 2 to 24 C atoms, such as isopropylmyristate (Rilanit® IPM), isononanoic acid C16-18 alkyl ester (Cetiol® SN), 2-ethylhexyl palmitate (Cegesoft® 24), stearic acid-2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanite® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are further preferred nourishing oil bodies.

Furthermore, dicarboxylic acid esters, symmetrical, asymmetrical or cyclic esters of carbonic acid with fatty alcohols, tri fatty acid esters of saturated and/or unsaturated linear and/or branched fatty acids with glycerol or fatty acid partial glycerides, which are understood to mean monoglycerides, diglycerides and industrial mixtures thereof, are suitable as nourishing ingredients.

Emulsifiers or surface-active agents are also preferably contained in the cosmetic products as contemplated herein. PEG derivatives of hydrogenated castor oil which are obtainable for example under the name PEG Hydrogenated Castor Oil are preferred, for example PEG-30 Hydrogenated Castor Oil, PEG-33 Hydrogenated Castor Oil, PEG-35 Hydrogenated Castor Oil, PEG-36 Hydrogenated Castor Oil, or PEG-40 Hydrogenated Castor Oil. The use of PEG-40 Hydrogenated Castor Oil is preferred as contemplated herein. These are preferably contained in a total amount of from about 0.05 to about 1.5 wt. %, preferably from about 0.1 to about 1.0 wt. %, preferably from about 0.2 to about 0.8 wt. %, in particular from about 0.3 to about 0.6 wt. %, in relation to the total weight of the cosmetic product.

The cosmetic products of the present disclosure can be formulated in the ways usual for the temporary reshaping of keratin fibers, in particular hair, for example as a hair gel, hairspray, hair mousse or hair wax. Formulation as a hair gel is preferred.

Both hair mousses and hairsprays require the presence of propellants and are formulated as what are known as aerosols. Vessels made of metal (aluminum, tinplate, tin), protected plastic or non-splintering plastic, or glass coated externally with plastic are potential pressure-resistant containers for aerosols of this type, wherein the compressive strength, breaking strength, corrosion resistance, ease of filling and also aesthetic considerations, ease of handling, printability, etc. play a role when selecting such a vessel. Special internal protective coatings ensure resistance to corrosion in respect of the cosmetic product as contemplated herein disposed in the pressure container. The used valves particularly preferably have an internally coated valve disk, wherein the coating and valve material are compatible with one another. If aluminum valves are used, the valve disks thereof can be coated internally, for example with Micoflex. If tinplate valves are used as contemplated herein, the valve disks thereof can be coated internally for example with PET (polyethylene terephthalate). With a given spraying device, the sizes of the aerosol droplets and the size distribution can be adjusted via the ratio of propellant to the other constituents of the cosmetic products.

If the cosmetic product as contemplated herein contains a propellant, this is contained advantageously in a total amount of from about 10 to about 80 wt. %, preferably from about 20 to about 70 wt. %, in particular from about 30 to about 60 wt. %, in relation to the total weight of the cosmetic product.

Propellants that are suitable as contemplated herein are selected for example from N₂O dimethylether, CO₂ air, alkanes having 3 to 5 carbon atoms, such as propane, n-butane, iso-butane, n-pentane and iso-pentane, and mixtures thereof. Dimethylether, propane, n-butane, iso-butane and mixtures thereof are preferred. The specified alkanes, or mixtures of the specified alkanes, or mixtures of the specified alkanes with dimethylether are used as sole propellant in accordance with a preferred embodiment. The present disclosure, however, also expressly comprises the co-use of propellants of the chlorofluorocarbon type, but in particular of the fluorinated hydrocarbon type.

Dimethylether or mixtures of propane and butane are very particularly preferably used as sole propellant in a ratio by weight of propane to butane of from about 20 to about 80 up to from about 15 to about 85. The mixtures are in turn preferably used in the compositions as contemplated herein in a total amount of from about 30 to about 55 wt. % in relation to the total weight of the cosmetic product. As contemplated herein, “butane” is understood to mean n-butane, iso-butane and mixtures of n-butane and iso-butane.

A further subject of the present disclosure is a method for the temporary shaping of keratin fibers, wherein a cosmetic product as contemplated herein is applied to the keratin fibers and these are then brought into the desired form.

That which has already been said in respect of the cosmetic products as contemplated herein applies, mutatis mutandis, in respect of further preferred embodiments of the method as contemplated herein.

In addition, a further subject of the present disclosure is the use of a cosmetic product as contemplated herein for the temporary shaping of keratin fibers.

Lastly, a further subject of the present disclosure is the use of a cosmetic product as contemplated herein to improve the moisture resistance of temporarily shaped keratin fibers.

That which has already been said in respect of the cosmetic products as contemplated herein and the method as contemplated herein applies, mutatis mutandis, in respect of further preferred embodiments of the uses as contemplated herein.

The present disclosure is outlined in particular by the following points:
1. A cosmetic product for the temporary shaping of keratin fibers, containing, in a cosmetically acceptable carrier,
a) at least one copolymer A, comprising the following monomers a1) and a2) and a3);
a1) at least one N-alkyl acrylamide,
a2) at least one C₁₋₈-alkyl amino-C₂₋₈-alkyl acrylate and/or C₁₋₈-alkyl amino-C₂₋₈-alkyl(meth)acrylate,
a3) at least one further monomer, selected from acrylic acid, methacrylic acid, C₁₋₈ alkyl esters of acrylic acid and/or (meth)acrylic acid and C₂₋₈ hydroxy alkyl esters of acrylic acid and/or (meth)acrylic acid,
b) at least one cross-linked polyurethane-vinyl copolymer B, and
c) at least one alkaline compound.
2. The cosmetic product according to point 1, exemplified in that the copolymer A consists of the monomers a1) and a2) and a3).
3. The cosmetic product according to either one of points 1 or 2, exemplified in that the copolymer A contains from about 20 to about 80 wt. %, preferably from about 30 to about 50 wt. % of monomer a1) and from about 1 to about 30 wt. %, preferably from about 2 to about 10 wt. % of monomer a2), in each case in relation to the total weight of the copolymer A.
4. The cosmetic product according to any one of the preceding points, exemplified in that the copolymer A contains, as monomer a1), an N-alkyl acrylamide with a linear or branched C₁₋₂₀ alkyl group, preferably with a linear or branched C₂₋₁₈ alkyl group, preferably with a linear or branched C₄₋₁₆ alkyl group, in particular with a linear or branched C₆₋₁₂ alkyl group.
5. The cosmetic product according to any one of the preceding points, exemplified in that the copolymer A contains N-tert-octylacrylamide as monomer a1).
6. The cosmetic product according to any one of the preceding points, exemplified in that the copolymer A contains, as monomer a2) a C₁₋₆-alkyl amino-C₂₋₆-alkyl acrylate and/or C₁₋₆-alkyl amino-C₂₋₆-alkyl(meth)acrylate, preferably a C₁₋₄-alkyl amino-C₂₋₄-alkyl acrylate and/or C₁₋₄-alkyl amino-C₂₋₄-alkyl(meth)acrylate, in particular tert-butylaminoethyl acrylate.
7. The cosmetic product according to any one of the preceding points, exemplified in that the copolymer A contains, as monomers a3), acrylic acid and/or methyl(meth)acrylate and/or hydroxypropyl acrylate.
8. The cosmetic product according to any one of the preceding points, exemplified in that the copolymer A contains from about 30 to about 50 wt. % of N-tert-octylacrylamide, from about 2 to about 10 wt. % of tert-butylaminoethyl acrylate, from about 10 to about 20 wt. % of acrylic acid and/or (meth)acrylic acid, from about 30 to about 40 wt. % of methyl(meth)acrylate, and from about 2 to about 10 wt. % of hydroxypropyl acrylate, in each case in relation to the total weight of the copolymer A.
9. The cosmetic product according to any one of the preceding points, exemplified in that the at least one copolymer A is contained in a total amount of from about 0.5 to about 10 wt. %, preferably from about 1.0 to about 8.0 wt. %, preferably from 1.0 to 6.0 wt. %, in particular from about 1.0 to about 5.0 wt. %, in relation to the total weight of the cosmetic product.
10. The cosmetic product according to any one of the preceding points, exemplified in that the cross-linked polyurethane-vinyl copolymer B is contained in the form of an aqueous particle dispersion having a mean particle size D₅₀ of from about 20 to about 200 nm, preferably from about 40 to about 150 nm, preferably from about 50 to about 120 nm, in particular from about 65 to about 90 nm.
11. The cosmetic product according to any one of the preceding points, exemplified in that the cross-linked polyurethane-vinyl copolymer B contains, as polyurethane polymer, a polymer from hexylene glycol, neopentyl glycol, adipic acid, methylene diphenyl diisocyanate and dimethyl propionic acid.
12. The cosmetic product according to any one of the preceding points, exemplified in that the cross-linked polyurethane-vinyl copolymer B contains, as vinyl polymer, a polymer from C₁₋₆ alkyl esters of acrylic acid and/or (meth)acrylic acid, vinyl acetate, vinyl chloride, styrene, isoprene, butadiene and acrylonitrile, in particular C₁₋₆ alkyl esters of acrylic acid and/or (meth)acrylic acid.
13. The cosmetic product according to any one of the preceding points, exemplified in that the cross-linked polyurethane-vinyl copolymer B contains, as vinyl polymer, a polymer from methyl(meth)acrylate.
14. The cosmetic product according to any one of the preceding points, exemplified in that the at least one cross-linked polyurethane-vinyl copolymer B is contained in a total amount of from about 0.1 to about 8.0 wt. %, preferably from about 0.2 to about 6.0 wt. %, preferably from about 0.4 to about 5.0 wt. %, more preferably from about 0.8 to about 4.0 wt. %, in particular from about 1.2 to about 3.2 wt. %, in relation to the total weight of the cosmetic product.
15. The cosmetic product according to any one of the preceding points, exemplified in that the cosmetic product has a ratio by weight of the total amount of the at least one copolymer A to the total amount of the at least one cross-linked polyurethane-vinyl copolymer B of from about 25:1 to about 1:20, preferably from about 10:1 to about 1:10, preferably from about 4:1 to about 1:3, in particular from about 2:1 to about 1:2.
16. The cosmetic product according to any one of the preceding points, exemplified in that the alkaline compound c) is selected from the group formed from 2-aminoethan-1-ol (monoethanolamine), tris(2-hydroxyethyl)-amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 2-amino-2-methylpropan-1-ol, 1-amino-2-methylpropan-2-ol, 3-aminopropan-1,2-diol, 2-amino-2-methylpropan-1,3-diol, in particular 2-amino-2-methylpropan-1-ol.
17. The cosmetic product according to any one of the preceding points, exemplified in that the alkaline compound c) is contained in a total quantity of from about 0.05 to about 7.0 wt. %, preferably from about 0.1 to about 5.0 wt. %, preferably from about 0.1 to about 4.0 wt. %, in particular from about 0.1 to about 3.0 wt. %, in relation to the total weight of the cosmetic product.
18. The cosmetic product according to any one of the preceding points, exemplified in that the cosmetic product additionally contains at least one further film-forming and/or firming polymer which is different from the copolymer A and the cross-linked polyurethane-vinyl copolymer B.
19. A method for the temporary shaping of keratin fibers, wherein a cosmetic product according to any one of points 1 to 18 is applied to the keratin fibers and these are then brought into the desired form.
20. Use of a cosmetic product according to any one of points 1 to 18 for the temporary shaping of keratin fibers.
21. Use of a cosmetic product according to any one of points 1 to 18 to improve the moisture resistance of temporarily shaped keratin fibers.

The following examples will explain the present disclosure, but are not intended to be limiting:

### EXAMPLES

### 1. Formulations (All Values Specified in wt. %, in Relation to the Total Weight of the Cosmetic Product in Question):

| Raw material | V1 | V2 | E1 |
|---|---|---|---|
| Amphomer (Copolymer A)¹⁾ | 5.0 | — | 2.5 |
| Hybridur 875 Polymer (Copolymer B)²⁾ | — | 12.5 | 6.25 |
| AMP-Ultra PC 2000 (alkaline compound)³⁾ | 0.9 | — | 0.46 |
| Water | to 100 | to 100 | to 100 |

¹⁾INCI name: Octylacrylamide/Acrylates/Butylaminoethyl Methacrylate Copolymer (National Starch)
²⁾INCI name: Polyurethane-2 and Polymethyl Methacrylate (30 wt. % dispersion in water, Air Products)

The cosmetic products V1, V2 and E1 were obtained by mixing the above ingredients. The polymer content in the cosmetic products V1, V2 and E1 was 5.0 wt. % in each case.

The moisture resistance of the prepared cosmetic products was tested by employing the HHCR test (high humidity curl retention test).

For this purpose, 10 strands of hair (European natural, type 827560 attached on one side, not glued, colour 6/0, length 240 mm, weight ˜0.6 g, Lₘₐₓ=220 mm, Kerling Internationale Haarfabrik GmbH) were prepared for each of the cosmetic products V1, V2 and E1 by applying 180 mg of the cosmetic product in question to the hair strands and massaging this in by hand. The strands of hair were then wound around rollers (length 160 mm, diameter 10 mm) and dried overnight at 298 K and 50% relative humidity. After unwinding the strands of hair, these were fixed to a metal frame, placed in a climatic chamber at 298 K and 85% relative humidity, and the length of the strands was determined directly after by employing a laser (corresponds to the value L₀). After a further 6 h the length of the strands of hair was determined again (corresponds to value Lₜ).

The moisture resistance, i.e. the HHCR value, was determined on the basis of the measured values in accordance with the following equation:

$$HHCR = \frac{{L}_{max} - {L}_{t}}{{L}_{max} - {L}_{0}} * 100$$

The results were examined by employing the Mann Whitney U test and were significant.

The HHCR values for the cosmetic products V1, V2 and E1 are specified in the following table, wherein the anticipated value E(V1+V2) corresponds to the mean value of the HHCR values of the two individual copolymers A and B.

| | V1 | V2 | E (V1 + V2) | E1 |
|---|---|---|---|---|
| HHCR [%] | 56 | 71 | 64 | 97 |

The HHCR value determined for the combination as contemplated herein (cosmetic product El) is thus significantly higher than the HHCR value anticipated for this combination (E(V1+V2)). Due to the use of the combination as contemplated herein of a copolymer A based on acrylates with a cross-linked polyurethane-vinyl copolymer B, a synergistic increase in the moisture resistance of cosmetic products for the temporary shaping of keratin fibers, in particular human hair, is achieved.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product for the temporary shaping of keratin fibers, comprising, in a cosmetically acceptable carrier,
a) at least one copolymer A, comprising the following monomers a1) and a2) and a3);
a1) at least one N-alkyl acrylamide,
a2) at least one C₁₋₈-alkyl amino-C₂₋₈-alkyl acrylate and/or C₁₋₈-alkyl amino-C₂₋₈-alkyl(meth)acrylate, and
a3) at least one further monomer, selected from acrylic acid, methacrylic acid, C₁₋₈ alkyl esters of acrylic acid and/or (meth)acrylic acid and C₂₋₈ hydroxy alkyl esters of acrylic acid and/or (meth)acrylic acid;
b) at least one cross-linked polyurethane-vinyl copolymer B; and
c) at least one alkaline compound.

2. The cosmetic product according to claim 1, wherein the copolymer A comprises, as monomer a1), an N-alkyl acrylamide with a linear or branched C₁₋₂₀ alkyl group.

3. The cosmetic product according to claim 1, wherein the copolymer A comprises, as monomer a2) a C₁₋₆-alkyl amino-C₂₋₆-alkyl acrylate and/or C₁₋₆-alkyl amino-C₂₋₆-alkyl(meth)acrylate.

4. The cosmetic product according to claim 1, wherein the copolymer A comprises, as monomers a3), acrylic acid and/or methyl(meth)acrylate and/or hydroxypropyl acrylate.

5. The cosmetic product according to claim 1, wherein the at least one copolymer A is comprised in a total amount of from about 0.5 to about 10 wt. %.

6. The cosmetic product according to claim 1, wherein the cross-linked polyurethane-vinyl copolymer B comprises, as polyurethane polymer, a polymer from hexylene glycol, neopentyl glycol, adipic acid, methylene diphenyl diisocyanate and dimethyl propionic acid.

7. The cosmetic product according to claim 1, wherein the cross-linked polyurethane-vinyl copolymer B comprises, as vinyl polymer, a polymer from methyl(meth)acrylate.

8. The cosmetic product according to claim 1, wherein the at least one cross-linked polyurethane-vinyl copolymer B is comprised in a total amount of from about 0.1 to about 8.0 wt. %.

9. A method for the temporary shaping of keratin fibers, the method comprising:
applying a cosmetic product according to claim 1 to the keratin fibers; and
bringing the keratin fibers into a desired form.

10. The cosmetic product according to claim 1, wherein the cosmetic product is utilized for the temporary shaping of keratin fibers.

11. The cosmetic product according to claim 1, wherein the copolymer A consists of the monomers a1) and a2) and a3).

12. The cosmetic product according to claim 1, wherein the copolymer A comprises N-tert-octylacrylamide as monomer a1).

13. The cosmetic product according to claim 1, wherein the copolymer A comprises tert-butylaminoethyl acrylate as monomers a2).

14. The cosmetic product according to claim 1, wherein the copolymer A comprises from about 30 to about 50 wt. % of N-tert-octylacrylamide, from about 2 to about 10 wt. % of tert-butylaminoethyl acrylate, from about 10 to about 20 wt. % of acrylic acid and/or (meth)acrylic acid, from about 30 to about 40 wt. % of methyl(meth)acrylate, and from about 2 to about 10 wt. % of hydroxypropyl acrylate, in each case in relation to the total weight of the copolymer A.

15. The cosmetic product according to claim 1, wherein the cross-linked polyurethane-vinyl copolymer B is contained in the form of an aqueous particle dispersion having a mean particle size D₅₀ of from about 20 to about 200 nm.

16. The cosmetic product according to claim 1, wherein the cross-linked polyurethane-vinyl copolymer B comprises, as vinyl polymer, a polymer from C₁₋₆ alkyl esters of acrylic acid and/or (meth)acrylic acid, vinyl acetate, vinyl chloride, styrene, isoprene, butadiene and acrylonitrile.

17. The cosmetic product according to claim 1, wherein the cosmetic product has a ratio by weight of the total amount of the at least one copolymer A to the total amount of the at least one cross-linked polyurethane-vinyl copolymer B of from about 25:1 to about 1:20.

18. The cosmetic product according to claim 1, wherein the alkaline compound c) is selected from the group of 2-aminoethan-1-ol (monoethanolamine), tris(2-hydroxyethyl)-amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 2-amino-2-methylpropan-1-ol, 1-amino-2-methylpropan-2-ol, 3-aminopropan-1,2-diol, 2-amino-2-methylpropan-1,3-diol, and combinations thereof.

19. The cosmetic product according to claim 18, wherein the alkaline compound c) is 2-amino-2-methylpropan-1-ol.

20. The cosmetic product according to claim 1, wherein the alkaline compound c) is comprised in a total quantity of from about 0.05 to about 7.0 wt. % in relation to the total weight of the cosmetic product.
